(19) United States
(12) Patent Application Publication
(10) Pub. No.: US 2021/0298830 A1
(43) Pub. Date: Sep. 30, 2021

(54) ROBOTIC SURGICAL SYSTEM AND METHODS OF USE THEREOF

(71) Applicant: Covidien LP, Mansfield, MA (US)

(72) Inventors: Amanda H. Lennartz, Erie, CO (US); Kenlyn S. Bonn, Lakewood, CO (US); Tyler J. Bagrosky, Arvada, CO (US)

(21) Appl. No.: 17/184,950

(22) Filed: Feb. 25, 2021

Related U.S. Application Data
(60) Provisional application No. 62/994,389, filed on Mar. 25, 2020.

(51) Int. Cl. A61B 34/10; A61B 34/00; A61B 34/30

(52) U.S. Cl. CPC A61B 34/10; A61B 2034/107; A61B 34/30; A61B 34/25

(57) ABSTRACT

A robotic surgical system including a robotic arm, a surgical instrument, a display, an input device, and a controller. The controller is in communication with the surgical instrument, the display, and the input device. The surgical instrument is configured to be coupled to the robotic arm. The display is configured to display an image of an area of interest of a surgical site. The input device is configured to be manipulated by a clinician. The controller is configured to generate a 3D model of the area of interest of the surgical site; display the 3D model on the display; generate a virtual surgical cut line corresponding to the movement of the input device relative to the 3D model; overlay the virtual surgical cut line on the 3D model; and cause the surgical instrument to cut the area of interest along a surgical cut line corresponding to the virtual surgical cut line with the surgical instrument.

### CROSS REFERENCE TO RELATED APPLICATION

This application claims the benefit of the filing date of provisional U.S. Application No. 62/994,389, filed on Mar. 25, 2020.

### FIELD

The present technology is generally related to the field of surgical systems, and more particularly to robotic surgical systems.

### BACKGROUND

Robotic surgical systems are increasingly becoming an integral part of minimally-invasive surgical procedures. Generally, robotic surgical systems include a surgeon console located remote from one or more robotic arms to which surgical instruments and/or cameras are coupled. Typically, a user provides inputs to the surgeon console, which are communicated to a central controller that translates the inputs into commands for telemanipulating the robotic arms, surgical instruments, and/or cameras during the surgical procedure.

### SUMMARY

In one aspect, the present disclosure provides a robotic surgical system including a robotic arm, a surgical instrument, a display, an input device, and a controller in communication with the surgical instrument, display, and input device. The surgical instrument is configured to be coupled to the robotic arm. The display is configured to display an image of an area of interest of a surgical site. The input device is configured to be manipulated by a clinician. The controller is configured to generate a 3D model of the area of interest of the surgical site; display the 3D model on the display; generate a virtual surgical cut line corresponding to the movement of the input device relative to the 3D model; overlay the virtual surgical cut line on the 3D model; and cause the surgical instruments to cut the area of interest along a surgical cut line corresponding to the virtual surgical cut line.

In aspects, the controller may be configured to determine whether the virtual surgical cut line intersects with a critical structure.

In aspects, the robotic surgical system may further include a sensor operably coupled to the controller and configured to identify the critical structure.

In aspects, the controller may be configured to overlay, on the 3D model, a location of the critical structure.

In aspects, the controller may be configured to indicate, on the display, that the virtual surgical cut line intersects with the critical structure.

In aspects, the controller may be configured to disable the surgical instrument upon determining that the virtual surgical cut line intersects with the critical structure.

In another aspect, the disclosure provides a method of performing a robotic surgical procedure. The method includes generating a 3D model of an area of interest of a surgical site; displaying the 3D model on a display of a robotic surgical system; generating a virtual surgical cut line corresponding to a movement of an input device of the robotic surgical system relative to the 3D model; overlaying the virtual surgical cut line on the 3D model; and causing an electrosurgical instrument operably coupled with the robotic surgical system to cut the area of interest along a surgical cut line corresponding to the virtual surgical cut line.

In aspects, generating the virtual surgical cut line may include delineating a desired surgical cut line on the area of interest.

In aspects, overlaying the virtual surgical cut line includes displaying the 3D model with the virtual surgical cut line on the display.

In aspects, the method may further include determining whether the virtual surgical cut line intersects with a critical structure.

In aspects, the method may further include overlaying, on the 3D model, a location of the critical structure.

In aspects, the method may further include highlighting, on the display, a portion of the virtual surgical cut line that intersects with the critical structure.

In aspects, the area of interest may be an organ, and the critical structure may be an artery or a vein within the organ.

In aspects, the method may further include disabling the electrosurgical instrument upon determining that the virtual surgical cut line intersects with the critical structure.

In another aspect, the disclosure provides a method of performing a robotic surgical procedure. The method includes generating a 3D model of an area of interest of a surgical site; displaying the 3D model on a display of a robotic surgical system; identifying a critical structure in the 3D model; highlighting the critical structure in the 3D model on the display; generating a virtual surgical cut line corresponding to a movement of an input device of the robotic surgical system relative to the 3D model; overlaying the virtual surgical cut line on the 3D model; causing an electrosurgical instrument operably coupled to the robotic surgical system to cut the area of interest along a surgical cut line, corresponding to the virtual surgical cut line.

In aspects, the method may further include disabling the electrosurgical instrument upon determining that the virtual surgical cut line intersects with the critical structure.

In aspects, the method may further include generating a new virtual surgical cut line after determining that the virtual surgical cut line intersects with the critical structure; and causing the electrosurgical instrument to cut the area of interest along the new virtual surgical cut line

In aspects, the area of interest is an organ.

In aspects, the critical structure is an artery or a vein within the organ.

In aspects, highlighting the critical structure in the 3D model on the display includes changing the color of the virtual surgical cut line that intersects with the critical structure.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic illustration of a robotic surgical system including a robotic surgical assembly in accordance with the present disclosure; and

FIG. 2 is a flow chart illustrating a method of using the robotic surgical system of FIG. 1.

### DETAILED DESCRIPTION

Embodiments of the presently disclosed robotic surgical system are described in detail with reference to the drawings, in which like reference numerals designate identical or corresponding elements in each of the several views. As used herein, the term “distal” refers to that portion of the robotic surgical system or component thereof, farther from the user, while the term “proximal” refers to that portion of the robotic surgical system, or component thereof, closer to the user.

As used herein, the terms parallel and perpendicular are understood to include relative configurations that are substantially parallel and substantially perpendicular up to about + or −10 degrees from true parallel and true perpendicular.

As used herein, the term “clinician” refers to a doctor, nurse, surgeon, or other care provider and may include support personnel. In the following description, well-known functions, or construction are not described in detail to avoid obscuring the disclosure in unnecessary detail.

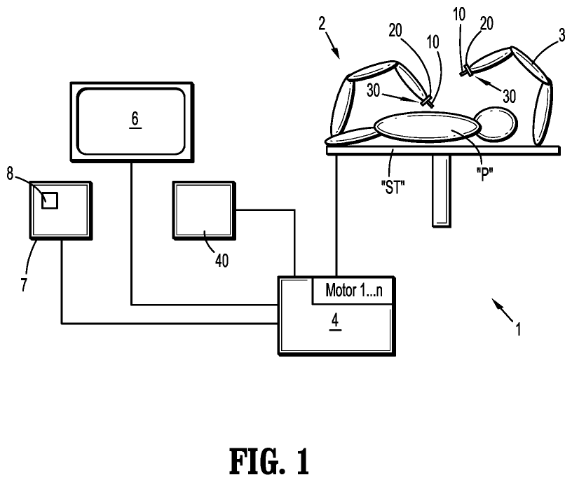

Referring initially to FIG. 1, a robotic surgical system 1 generally includes a plurality of surgical robotic arms 2, 3, a controller 4, a display device 6, an input device 7, a robotic assembly 30, and an image capture device 40. The display device 6 is configured to be coupled to the controller 4.

The robotic assembly 30 generally includes a surgical instrument 10 configured to be removably attached to an instrument drive unit 20 of one or both of robotic arms 2, 3. The surgical instrument 10 may be any suitable instrument for performing a surgical procedure, such as, for example, a uterine manipulator or a tissue resecting instrument. In aspects, the surgical instrument 10 may be a surgical stapling device, an electrosurgical instrument, an ultrasonic dissector, a mechanical dissector, or any other suitable surgical instrument.

The image capture device 40 may be any type of real-time imaging system, such as, for example magnetic resonance imaging (MRI), ultrasound, computed tomography (CT), position emission tomography (PET), etc., for capturing images and/or video of a surgical site inside the patient “P”. The image capture device 40 is configured to couple to the controller 4 and transmit captured imaging data to the controller 4, which creates three-dimensional (3D) image of inside the patient “P” in real-time and transmits the 3D image to the display 6.

The input device 7 is coupled to the controller 4 and configured such that a clinician may telemanipulate the robotic arms 2, 3 in a first operating mode. The input device 7 is configured to be coupled to the controller 4. The input device 7 may further include a sensor 8 configured to track the movement of the input device 7 relative to an image displayed on the display 6. Additionally or alternatively, the input device 7 may include a camera (not shown) to track the movement of the input device 7 relative to an image displayed on the display 6. In some embodiments, the input device 7 may communicate with the controller 4 wirelessly.

Each of the robotic arms 2, 3 may include a plurality of segments, which are connected through joints. The robotic arms 2, 3 may be driven by electric drives (not shown) that are connected to the controller 4. The controller 4 (e.g., a computer) is set up to activate the drives, in particular by means of a computer program, in such a way that the robotic arms 2, 3, their instrument drive units 20, and thus the surgical instrument 10 executes a desired movement according to a movement defined by means of the input device 7. The controller 4 may also be set up in such a way that it regulates the movement of the robotic arms 2, 3 and/or of the drives.

The robotic surgical system 1 is configured for use on a patient “P” lying on a surgical table “ST” to be treated in a minimally invasive manner by means of a surgical instrument. The robotic surgical system 1 may also include more than two robotic arms 2, 3, the additional robotic arms likewise being connected to the controller 4 and being telemanipulatable by means of the input device 7. The surgical instrument 10, for example, the uterine manipulator and/or tissue resecting instrument, may also be attached to the additional robotic arm.

The controller 4 may control a plurality of motors (Motor 1 . . . n) with each motor configured to drive the surgical instrument 10 to effect operation and/or movement of components of the surgical instrument 10. It is contemplated that the controller 4 coordinates the activation of the various motors (Motor 1 . . . n) to coordinate a clockwise or counter-clockwise rotation of drive members of the instrument drive unit 20 in order to coordinate an operation and/or movement of a respective component of the surgical instrument 10.

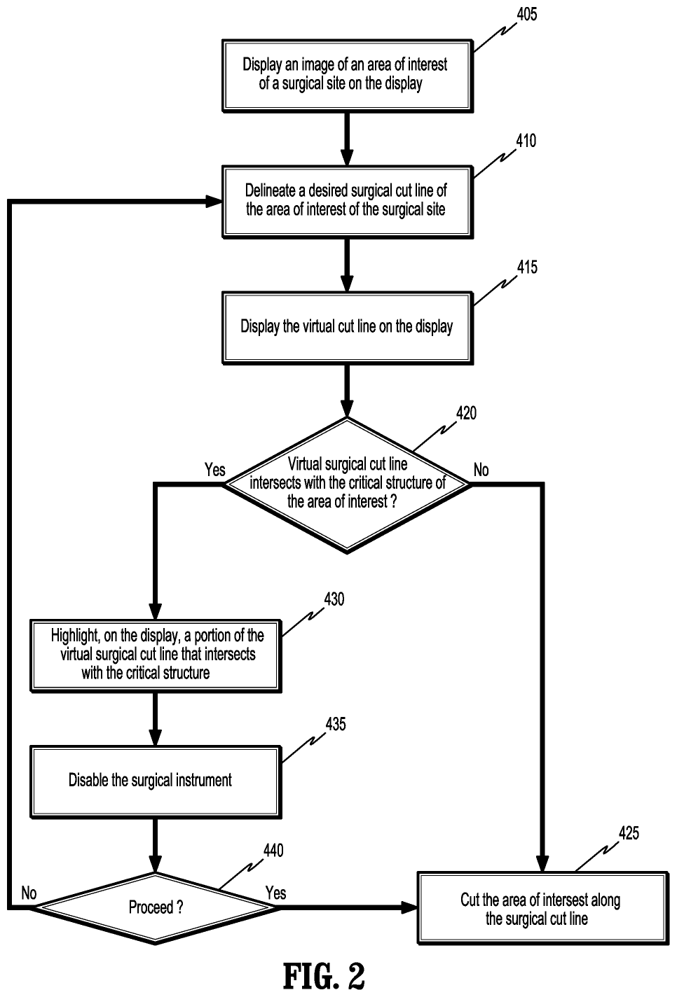

With reference to FIG. 2, a method of performing a surgical procedure using the robotic surgical system 1 will now be described. The surgical procedure may be a sleeve gastrectomy, a hysterectomy, or any other suitable procedure. In step 405 of the method, an image of an area of interest in the surgical site (e.g., an organ) is displayed on the display 6. In particular, pre-procedure images of the surgical site inside patient “P” are obtained using the image capture device 40. The images and/or video captured by the image capture device 40 are transmitted to the controller 4, which is utilized to generate and display on the display 6 a three-dimensional (3D) model of the surgical site.

In step 410, the clinician delineates a desired surgical cut line on the 3D model. In particular, the clinician moves the input device 7 relative to the displayed 3D model of the area of interest along a desired surgical cut line. In response to movement of the input device 7, in step 415, the controller 4 generates and overlays and/or superimposes the virtual surgical cut line over the displayed 3D model of the area of interest. In aspects, the desired surgical cut line may be a desired seal line, staple line, resection line, or transection line.

In some embodiments, prior to or after the clinician delineates the virtual surgical cut line, the area of interest may be assessed for any critical structures (e.g., a vile duct, gall bladder, lymph vessels, arteries, and/or veins). To identify the presence and location of any critical structures in the area of interest, the robotic surgical system 1 may include sensors configured to sense physical characteristics or physiological attributes of tissue, e.g., contents, physical density, acoustic density, thermal properties of the tissue, etc., that are indicative of a critical structure. The identified locations of the critical structures are overlaid on the 3D model to assist the clinician in determining where to make the virtual surgical cut line. After identifying the locations of the critical structures, the controller 4 may determine an optimal cut line that avoids the critical structures and overlays the optimal cut line on the 3D model to assist the clinician in drawing the virtual surgical cut line.

In aspects, the virtual surgical cut line may be made by the robotic surgical system 1 rather than the clinician, with the clinician having final approval of the virtual surgical cut line made by the robotic surgical system 1. The robotic surgical system 1 may be configured to make and display an alternative virtual surgical cut line in response to the clinician denying the initial virtual surgical cut line generated by the robotic surgical system 1.

After the clinician delineates the virtual surgical cut line on the 3D model, in step 420, the controller 4 determines whether the virtual surgical cut line intersects with any portion of the identified critical structure. Upon determining that the virtual surgical cut line does intersect with the critical structure, in step 430, the controller 4 highlights on the display 6 the portion of the virtual surgical cut line that intersects with the critical structure. Additionally or alternatively, the controller 4 may highlight the portion of the critical structure. Highlighting may include the 3D model changing the color of the entire virtual surgical cut line to red or any other suitable color indicative of an error.

In step 435, in response to determining that the virtual surgical cut line intersects with the critical structure, the controller 4 may disable the surgical instrument 10 to prevent activation of the surgical instrument 10 by the clinician. In some embodiments, in response to determining that the virtual surgical cut line intersects with a critical structure, the clinician may receive an alert, such as an audible warning or a flashing on the display 6. In step 440, once the clinician is alerted, the clinician may decide to proceed with the surgical procedure or delineate a new surgical cut line that avoids the identified critical structure.

In step 445, if it is determined that the virtual surgical cut line does not intersect with any critical structures, the surgical instrument 10 of the robotic surgical system 1 cuts the area of interest along a surgical cut line that corresponds to the virtual surgical cut line delineated by the clinician. In aspects, the robotic surgical system 1 may perform the cut automatically after determining that no critical structures intersect with the virtual surgical cut line. In other aspects, the clinician may prompt the robotic surgical system 1 to perform the cut after the clinician determines, using the aid of the 3D model, that the virtual surgical cut line does not intersect with a critical structure.

In aspects, the robotic arms 2, 3 and/or the surgical instrument 10 may further include a plurality of sensors (e.g., position sensors, orientation sensors, accelerometers, etc.) to assist in determining position and orientation of various joints of the robotic arms 2, 3 and various components of the surgical instrument 10. For example, during use of the uterine manipulator, a position of each of the joints and various components of the uterine manipulator relative to the patient and patient's anatomy (e.g., sensitive surrounding structures) may be determined based on kinematics. The robotic surgical system 1 may determine the kinematics of the attached surgical instrument 10 to provide a size, a location, and/or a range of motion of the attached surgical instrument 10. Additionally, and or alternatively, the size, the location, and the range of motion of the attached surgical instrument 10 may be overlaid on the 3D model. The combination of the size, the location, the range of motion, and the virtual surgical cut line may provide the clinician with more information to avoid critical structures. For example, the robotic surgical system 1 may determine, based on the kinematics of the surgical instrument 10, that the surgical instrument 10 has a large throw length for articulation, and adjusts the virtual surgical cut line to accommodate the range of motion of the surgical instrument 10. In aspects, the robotic surgical system 1 may determine, based on the range of motion of the surgical instrument 10, whether the movement of the surgical instrument 10 would damage nearby critical structures. In aspects, the robotic surgical system 1 may adjust a length and/or width of the virtual surgical cut line based on the size of the surgical instrument 10.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based controller. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A robotic surgical system, comprising:
a robotic arm;
a surgical instrument configured to be coupled to the robotic arm;
a display configured to display an image of an area of interest of a surgical site;
an input device configured to be manipulated by a clinician; and
a controller in communication with the surgical instrument, the display, and the input device, the controller configured to:
generate a 3D model of the area of interest of the surgical site;
display the 3D model on the display;
generate a virtual surgical cut line corresponding to the movement of the input device relative to the 3D model;
overlay the virtual surgical cut line on the 3D model; and
cause the surgical instrument to cut the area of interest along a surgical cut line corresponding to the virtual surgical cut line.

2. The robotic surgical system according to claim 1, wherein the controller is configured to determine whether the virtual surgical cut line intersects with a critical structure.

3. The robotic surgical system according to claim 2, further comprising a sensor operably coupled to the controller and configured to identify the critical structure.

4. The robotic surgical system according to claim 2, wherein the controller is configured to overlay, on the 3D model, a location of the critical structure.

5. The robotic surgical system according to claim 4, wherein the controller is configured to indicate, on the display, that the virtual surgical cut line intersects with the critical structure.

6. The robotic surgical system according to claim 2, wherein the controller is configured to disable the surgical instrument upon determining that the virtual surgical cut line intersects with the critical structure.

7. A method of performing a robotic surgical procedure, the method comprising:
generating a 3D model of an area of interest of a surgical site;
displaying the 3D model on a display of a robotic surgical system;
generating a virtual surgical cut line corresponding to a movement of an input device of the robotic surgical system relative to the 3D model;
overlaying the virtual surgical cut line on the 3D model; and
causing an electrosurgical instrument operably coupled with the robotic surgical system to cut the area of interest along a surgical cut line corresponding to the virtual surgical cut line.

8. The method according to claim 7, wherein generating the virtual surgical cut line includes delineating a desired surgical cut line on the area of interest.

9. The method according to claim 7, wherein overlaying the virtual surgical cut line includes displaying the 3D model with the virtual surgical cut line on the display.

10. The method according to claim 7, further comprising determining whether the virtual surgical cut line intersects with a critical structure.

11. The method according to claim 10, further comprising overlaying, on the 3D model, a location of the critical structure.

12. The method according to claim 10, further comprising highlighting, on the display, a portion of the virtual surgical cut line that intersects with the critical structure.

13. The method according to claim 10, wherein the area of interest is an organ, and the critical structure is an artery or a vein within the organ.

14. The method according to claim 10, further comprising disabling the electrosurgical instrument upon determining that the virtual surgical cut line intersects with the critical structure.

15. A method of performing a robotic surgical procedure, the method comprising:
generating a 3D model of an area of interest of a surgical site;
displaying the 3D model on a display of a robotic surgical system;
identifying a critical structure in the 3D model;
highlighting the critical structure in the 3D model on the display;
generating a virtual surgical cut line corresponding to a movement of an input device of the robotic surgical system relative to the 3D model;
overlaying the virtual surgical cut line on the 3D model; and
causing an electrosurgical instrument operably coupled to the robotic surgical system to cut the area of interest along a surgical cut line corresponding to the virtual surgical cut line.

16. The method according to claim 15, further comprising disabling the electrosurgical instrument upon determining that the virtual surgical cut line intersects with the critical structure.

17. The method according to claim 15, further comprising:
generating a new virtual surgical cut line after determining that the virtual surgical cut line intersects with the critical structure; and
causing the electrosurgical instrument to cut the area of interest along the new virtual surgical cut line.

18. The method according to claim 15, wherein the area of interest is an organ.

19. The method according to claim 18, wherein the critical structure is an artery or a vein within the organ.

20. The method according to claim 15, wherein highlighting the critical structure in the 3D model on the display includes changing the color of the virtual surgical cut line that intersects with the critical structure.
